(19) United States
(12) Patent Application Publication
(10) Pub. No.: US 2024/0191273 A1
(43) Pub. Date: Jun. 13, 2024

(54) ANTHOCYANIN BIOPRODUCTION IN A CELL-FREE MANUFACTURING SYSTEM

(71) Applicant: DEBUT BIOTECHNOLOGY, INC., San Diego, CA (US)

(72) Inventors: Joshua Britton, San Diego, CA (US); Nicholas Brideau, San Diego, CA (US); Neill Kim, San Diego, CA (US)

(21) Appl. No.: 18/283,114

(22) PCT Filed: Apr. 5, 2022
(86) PCT No.: PCT/US2022/023497

§ 371 Date: Sep. 20, 2023

Related U.S. Application Data
(60) Provisional application No. 63/172,297, filed on Apr. 8, 2021.

(51) Int. Cl. C12P 19/44

(52) U.S. Cl. CPC C12P 19/445; C12Y 204/01091; C12Y 204/01185

(57) ABSTRACT

A cell-free system is provided to produce anthocyanins from anthocyanidins. An anthocyanidin is added to a reaction mixture comprising a glycosyl transferase enzyme and UDP-sugar. The reaction mixture may be aqueous and may comprise a co-solvent, e.g., an organic co-solvent, such as DMF.

### FIELD OF THE INVENTION

The present invention features a method of producing anthocyanins by enzyme-modification of anthocyanidins. In particular, the present invention features a cell-free production method.

### BACKGROUND OF THE INVENTION

Anthocyanins (glycosides of anthocyanidins) are major secondary metabolites found in plants that are responsible for color variation seen in fruit and flowers. The building blocks for anthocyanins are anthocyanidins, e.g., one of six major anthocyanidins: cyanidin, delphinidin, malvidin, pelargonidin, peonidin and petunidin, which are glycosylated by uridine-5′-diphosphate (UDP) carbohydrate-dependent glycosyltransferase (UGT) enzymes. Addition of various nucleotide activated sugars (i.e., arabinose, galactose, glucose, xylose, rhamnose) in different combinations of type, number, and position contribute to the diversity of anthocyanins found in nature.

Industrial uses of anthocyanins are focused on their color properties as well as potential health benefits. With an increasing shift away from artificial dyes towards natural ingredient equivalents for food, textiles, and cosmetics products, anthocyanins are becoming a popular class of molecules. Furthermore, years of research has gone into investigating the anti-oxidative, anti-inflammatory, anti-cancer, anti-obesity, cardio- and neuroprotective properties of different anthocyanins.

Manufacturing anthocyanins via cultivation, chemical synthesis, or in cells suffers from many problems that limit the commercial viability of high-value chemical production. First, cultivation is often economically unfeasible, requires a vast amount of land/energy/water and the plant is only capable of producing the high-value material in low amounts. Next, chemical synthesis requires extensive, elaborate, expensive, toxic, and inefficient multi-step chemical reactions to produce natural products that often are too complex to make in the laboratory. Finally, bio-foundries (use of the whole cell) suffer from product toxicity, carbon flux redirection, diffusion problems through cell walls, and toxic byproduct generation. To avoid these above problems, cell-free manufacturing presents as a viable alternative.

In cell-free systems, the key components of the cell, namely cofactors and enzymes, are used in a chemical reaction without the cell. The same enzymes that are found in plants are created in vivo (typically through protein overexpression in hosts such as bacteria), isolated via chromatography, and then added into a bioreactor with a substrate (starting material). The enzymes transform the substrate in the same way that occurs in plants but without the complexity of the organism. In this way, natural products can begin to be created without the plant, cell, or chemical synthesis.

### BRIEF SUMMARY OF THE INVENTION

It is an objective of the present invention to provide a cell-free method that allows for the production of anthocyanidins (cyanidin glycosides, such as cyanidin-3-glycosides, cyanidin-5-glycosides, etc.) starting from anthocyanidins via a cell-free biosynthesis platform. Embodiments of the present invention can be freely combined with each other if they are not mutually exclusive.

The present invention uses glycotransferase enzymes to convert anthocyanidins to several possible anthocyanidin glycosides, such as cyanidin-3-glycosides, depending on the glycosyl donor.

The presently claimed process uses a controlled enzymatic step to increase product titer in short reaction times. In some embodiments, the controlled enzymatic step may increase product titer by a factor of at least five. Additionally, enzyme immobilization may be used to enhance anthocyanidin conversion to corresponding anthocyanins. For example, enzyme immobilization was used to improve cyanidin conversion to cyanidin-3-glucoside (C3G) by avoiding enzyme precipitation, inactivation, and the unreliability of the non-immobilized system.

A unique and inventive technical feature of the present invention is the use of a cell-free system for the production of anthocyanins (anthocyanidin glycosides), such as cyanidin-3-glycoside. Without wishing to limit the invention to any theory or mechanism, it is believed that the technical feature of the present invention advantageously provides for higher reaction concentrations of starting materials, reagents, and enzymes, resulting in higher concentrations of final products. Additionally, the present invention eliminates the complications of cell walls, thereby eliminating a barrier to product and substrate diffusion. Furthermore, the present invention eliminates competition for carbon flux, which limits the efficiency of cell-based synthesis methods, and thus greatly reduces byproduct formation. Also, because there is no cell, the present invention is not vulnerable to degradation by cell death due to the formation of toxic compounds. The adaptability of the system to accommodate different starting anthocyanidins and glycosylated UDPs (UDP-sugars) lends the system flexibility to produce a multitude of products and the ability to use various solvents, such as organic solvents, to permit higher concentrations of solutes without worrying about killing the cell.

The present invention is adaptable to various products. In this approach, one simply changes the anthocyanidin, UDP-sugar, and UGT enzyme in the pathway to synthesize the desired anthocyanin product.

Furthermore, prior references teach away from the present invention. The maximum titers achievable for current cell-based production of anthocyanins are limited. Previously published work involving extensive cellular reprogramming and metabolic engineering produce anthocyanins such as cyanidin-3-glucoside on the scale of 0-200 mg/L. Although there have been reports of microbial hosts producing titers approaching 300-400 mg/L, these experiments are either not technically reproducible (see, e.g., Shrestha, 2019), or contain data that is not calculated to a standard set forth in the field (Yan, 2008). The present invention affords a C3G production titer of at least 1.5 g/L, representing up to a 50-fold increase from reported values.

Furthermore, the inventive technical features of the present invention contributed to a surprising result that would not have been predicted from the cell-based synthesis literature. For example, the stability of the starting material cyanidin is dependent on the pH of the solution it is contained in, with the highest stability at pH<5.0. These low pH values are not compatible with cell-based production, as the optimum pH for cells is 7. The present invention is completely functional at a range of pH values (4.0-9.0). In some embodiments, it is preferred to carry out the reaction at a pH at or near the anthocyanidin's pH of maximum stability, for example, at or near pH 5 (e.g., 4.0-6.0, 4.2-5.8, 4.3-5.7, 4.4-5.6, 4.5-5.5, 4.6-5.4, 4.7-5.3, 4.8-5.2, or 4.9-5.1), in order to maximize the stability of the anthocyanidin.

Additionally, the solubility of the starting material anthocyanidin may be enhanced by using a solvent, such as an organic solvent, which the present invention permits. For example, the solubility of cyanidin is only 49 mg/L in water, and it will degrade within minutes at this concentration. Addition of a co-solvents such as dimethylformamide (DMF), dimethylsulfoxide (DMSO), methanol (MeOH), or ethanol (EtOH) (or mixtures of two or more thereof) greatly improves the solubility and stability of cyanidin for the reactions. Some methods according to the present invention can operate at co-solvent levels>60%—at least 12 times higher than cell-based anthocyanin production, as living cells are sensitive to most co-solvent concentrations above 5%.

The inventive technical features of the present invention contributed to a further unexpected result of permitting high concentrations of UDP-sugar in the reaction mixture. For example, the conversion of cyanidin to C3G requires a UDP-conjugated sugar donor molecule such as UDP-Glucose. The UDP-glucotransferase enzyme has low affinity for UDP-Glucose and the reactions are dependent on large molar excess of this molecule. The level of UDP-Glucose in the present invention is controllable and can vary from 1 mM-30 mM (0.5-17 g/L), compared to cell-based production which requires substantial cell engineering to reach UDP-Glucose levels approaching 2 g/L (Feng et al 2020).

Any feature or combination of features described herein are included within the scope of the present invention provided that the features included in any such combination are not mutually inconsistent as will be apparent from the context, this specification, and the knowledge of one of ordinary skill in the art. For example, while certain anthocyanins are presented as products of certain anthocyanidins, one skilled in the art will recognize that the methods described herein can be generalized to produce a wide range of anthocyanins from a range of anthocyanidins. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1A shows the structures of common anthocyanidins. The structure on the left is the anthocyanidin core structure and the table on the right shows the “Rn” substituents at the indicated positions of the anthocyanidin core structure.

FIG. 1B shows the chemical transformation pathway for the production of an anthocyanin (e.g., cyanidin-3-0-glucoside, a.k.a. C3G) from an anthocyanidin (e.g., cyanidin) and a UDP-sugar (e.g., UDP-glucose).

FIGS. 2A-2C Shows HPLC traces for cyanidin, cyanidin-3-glycoside standards (top, FIG. 2A), non-immobilized enzyme reactions (middle, FIG. 2B), and immobilized enzyme reactions (bottom, FIG. 2C). The retention times of 6.95 minutes (C3G) and 8.9 minutes (cyanidin) at 530nm are noted.

FIGS. 3A-3B show the schematic diagram of the continuous reactor (top, FIG. 3A), and HPLC traces for molecule standards as well as cyanidin-3-glycoside produced in the continuous reactor (bottom, FIG. 3B). The retention times of 6.95 minutes (C3G) and 8.9 minutes (cyanidin) at 530 nm are noted.

### DETAILED DESCRIPTION OF THE INVENTION

Before the present compounds, compositions, and/or methods are disclosed and described, it is to be understood that, unless specified, this invention is not limited to specific synthetic methods or to specific compositions, as such may, of course, vary. It is also to be understood that the terminology used herein is for the purpose of describing particular embodiments only and is not intended to be limiting.

As used herein, “reaction solution” may refer to all components necessary for enzyme-based chemical transformation of an anthocyanidin to an anthocyanin. This is typically, but not limited to, buffering agent, salts, cosolvent, cofactor, and substrate (starting material).

As used herein, “reaction mixture” may refer to all components from the “reaction solution” plus the enzyme(s) and/or products from the reaction. In some embodiments, the “reaction mixture” may refer to just the reaction solution without any enzymes or reaction products.

In some embodiments, “reaction solution” and “reaction mixture” may be used interchangeably.

As used herein, “buffering agents” may refer to chemicals added to water-based solutions that resist changes in pH by the action of acid-base conjugate components.

As used herein, “supernatant” may refer to the soluble liquid fraction of a sample.

As used herein, “batch reactions” may refer to a chemical or biochemical reaction performed in a closed system such as a fermenter or typical reaction flask.

As used herein, “cofactors” may refer to a non-protein chemical compound that may bind to a protein and assist with a biological chemical reaction. Non-limiting examples of cofactors may include but are not limited to UTP.

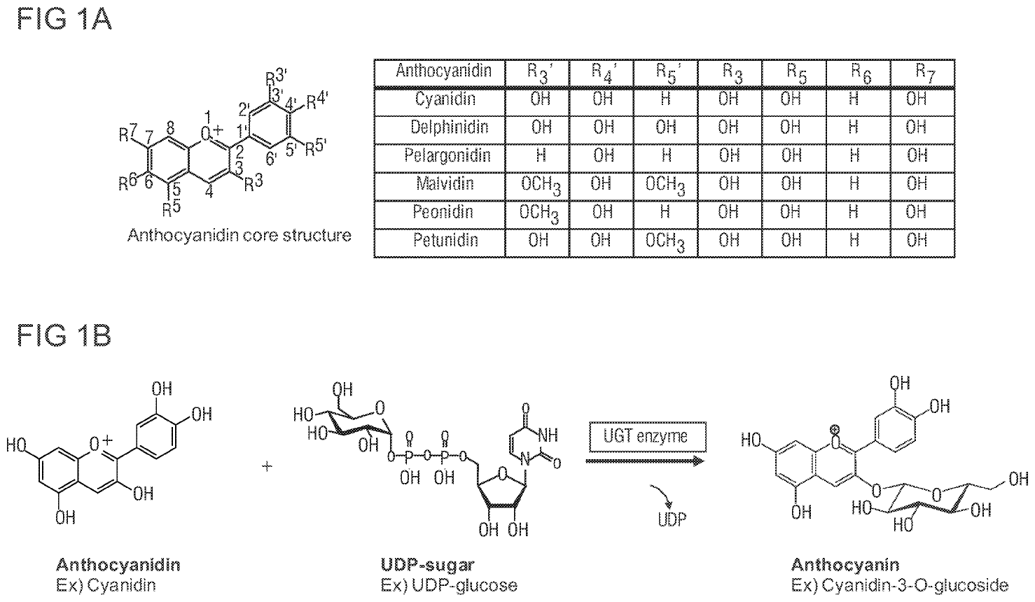

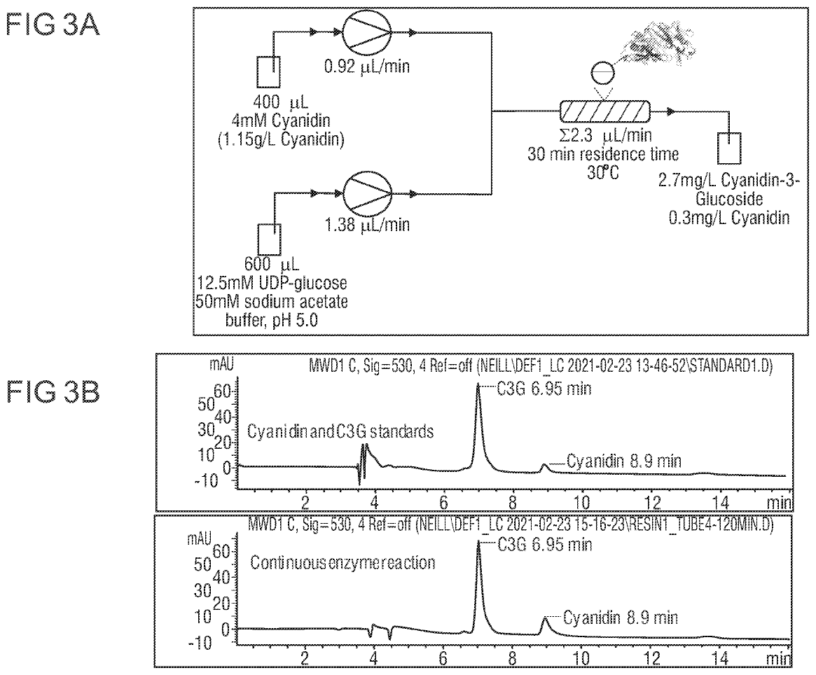

Referring now to FIGS. 1A-3B, the present invention is illustrated by reference to an exemplary method of producing glycosylated cyanidin from cyanidin.

The method illustrated in FIGS. 1A-3B can be generalized to incorporate any suitable combination of anthocyanidin, UTP-glycoside, and enzyme, to produce the desired product anthocyanin. For example, the anthocyanidin may be an anthocyanidin as set forth in Table 1:

TABLE 1
Common anthocyanidin starting materials.

| Structure | Anthocyanidin | R^{3′} | R^{4′} | R^{5′} | R³ | R⁵ | R⁶ | R⁷ |
|---|---|---|---|---|---|---|---|---|
| 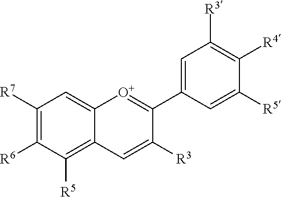 | Aurantinidin Cyanidin Delphinidin Europinidin Pelargonidin Malvidin Peonidin Petunidin Rosinidin | —H —OH —OH —OCH₃ —H —OCH₃ —OCH₃ —OH —OHC₃ | —OH —OH —OH —OH —OH —OH —OH —OH —OH | —H —H —OH —OH —H —OCH₃ —H —OCH₃ —H | —OH —OH —OH —OH —OH —OH —OH —OH —OH | —OH —OH —OH —OCH₃ —OH —OH —OH —OH —OH | —OH —H —H —H —H —H —H —H —H | —OH —OH —OH —OH —OH —OH —OH —OH —OCH₃ |

In some embodiments the UDP-conjugated sugar may vary. In some embodiments the sugar may be glucose, galactose, xylose, arabinose, or rhamnose.

In some embodiments the product may be cyanidin-3-glucoside (C3G). In some embodiments, the product may be any product listed in Table 2 or Table 3, infra.

In some embodiments, the temperature of the reaction may range from ab out 20° C. to about 50° C. In some embodiments, the temperature of the reaction is about 30° C.

In some embodiments, the pH of the reaction may range from about 4 to about 9.0. In some embodiments, the pH of the reaction is about 5.0 (e.g., 4.0-6.0, 4.2-5.8, 4.3-5.7, 4.4-5.6, 4.5-5.5, 4.6-5.4, 4.7-5.3, 4.8-5.2, or 4.9-5.1).

The reaction time may be varied to optimize yield or to balance yield against efficient use of resources. The reaction time may vary from 10 min. to 48 hours, e.g., from 15 min. to about 36 hours, or about 30 min. to 24 hours. In some embodiments, the time to run the reaction may range from about 30 min. to about 1 hour.

In some embodiments, the enzymes may be immobilized. In some embodiments, immobilized enzymes are immobilized onto solid supports. Non-limiting examples of solid supports may include but are not limited to epoxy methacrylate, amino C6 methacrylate, or microporous polymethacrylate. In further embodiments, various surface chemistries may be used for linking the immobilized enzyme to a solid surface, including but not limited to covalent, adsorption, ionic, affinity, encapsulation, or entrapment. In other embodiments, the enzymes are non-immobilized. Either immobilized or non-immobilized enzymes may be employed in batch or continuous synthesis. For example, an immobilized enzyme on a solid support may be used in a continuous flow cell through which a reaction mixture passes, whereby an immobilized enzyme may catalyze modification of substrate to produce the product at a high titer. Alternatively, a continuous method may comprise micro mixing of enzyme solution and substrate to produce the product at a high titer, while continuously removing product, removing substrate, or both.

The anthocyanins in Table 2 may be prepared from the corresponding anthocyanidins using 3-O-glycotransferase or 5-O-glycotransferase as the glycosylating enzyme.

TABLE 2
Anthocyanidin and glycosyl donor molecules and the corresponding anthocyanin
products using 3-O-glycotransferase (3GT) or 5-O-glycotransferase (5GT).

| Anthocyanidin | Enzyme | Glycosyl Donor Molecule | Anthocyanin |
|---|---|---|---|
| Cyanidin | 3GT | UDP-arabinose | cyanidin-3-O-arabinoside |
| Cyanidin | 3GT | UDP-galactose | cyanidin-3-O-galactoside |
| Cyanidin | 3GT | UDP-glucose | cyanidin-3-O-glucoside |
| Cyanidin | 3GT | UDP-rhamnose | cyanidin-3-O-rhamnoside |
| Cyanidin | 3GT | UDP-xylose | cyanidin-3-O-xyloside |
| Cyanidin | 5GT | UDP-arabinose | cyanidin-5-O-arabinoside |
| Cyanidin | 5GT | UDP-galactose | cyanidin-5-O-galactoside |
| Cyanidin | 5GT | UDP-glucose | cyanidin-5-O-glucoside |
| Cyanidin | 5GT | UDP-rhamnose | cyanidin-5-O-rhamnoside |
| Cyanidin | 5GT | UDP-xylose | cyanidin-5-O-xyloside |
| Delphinidin | 3GT | UDP-arabinose | delphinidin-3-O-arabinoside |
| Delphinidin | 3GT | UDP-galactose | delphinidin-3-O-galactoside |
| Delphinidin | 3GT | UDP-glucose | delphinidin-3-O-glucoside |
| Delphinidin | 3GT | UDP-rhamnose | delphinidin-3-O-rhamnoside |
| Delphinidin | 3GT | UDP-xylose | delphinidin-3-O-xyloside |
| Delphinidin | 5GT | UDP-arabinose | delphinidin-5-O-arabinoside |
| Delphinidin | 5GT | UDP-galactose | delphinidin-5-O-galactoside |
| Delphinidin | 5GT | UDP-glucose | delphinidin-5-O-glucoside |
| Delphinidin | 5GT | UDP-rhamnose | delphinidin-5-O-rhamnoside |
| Delphinidin | 5GT | UDP-xylose | delphinidin-5-O-xyloside |
| Malvidin | 3GT | UDP-arabinose | malvidin-3-O-arabinoside |
| Malvidin | 3GT | UDP-galactose | malvidin-3-O-galactoside |
| Malvidin | 3GT | UDP-glucose | malvidin-3-O-glucoside |
| Malvidin | 3GT | UDP-rhamnose | malvidin-3-O-rhamnoside |
| Malvidin | 3GT | UDP-xylose | malvidin-3-O-xyloside |
| Malvidin | 5GT | UDP-arabinose | malvidin-5-O-arabinoside |
| Malvidin | 5GT | UDP-galactose | malvidin-5-O-galactoside |
| Malvidin | 5GT | UDP-glucose | malvidin-5-O-glucoside |
| Malvidin | 5GT | UDP-rhamnose | malvidin-5-O-rhamnoside |
| Malvidin | 5GT | UDP-xylose | malvidin-5-O-xyloside |
| Pelargonidin | 3GT | UDP-arabinose | pelargonidin-3-O-arabinoside |
| Pelargonidin | 3GT | UDP-galactose | pelargonidin-3-O-galactoside |
| Pelargonidin | 3GT | UDP-glucose | pelargonidin-3-O-glucoside |
| Pelargonidin | 3GT | UDP-rhamnose | pelargonidin-3-O-rhamnoside |
| Pelargonidin | 3GT | UDP-xylose | pelargonidin-3-O-xyloside |
| Pelargonidin | 5GT | UDP-arabinose | pelargonidin-5-O-arabinoside |
| Pelargonidin | 5GT | UDP-galactose | pelargonidin-5-O-galactoside |
| Pelargonidin | 5GT | UDP-glucose | pelargonidin-5-O-glucoside |
| Pelargonidin | 5GT | UDP-rhamnose | pelargonidin-5-O-rhamnoside |
| Pelargonidin | 5GT | UDP-xylose | pelargonidin-5-O-xyloside |
| Peonidin | 3GT | UDP-arabinose | peonidin-3-O-arabinoside |
| Peonidin | 3GT | UDP-galactose | peonidin-3-O-galactoside |
| Peonidin | 3GT | UDP-glucose | peonidin-3-O-glucoside |
| Peonidin | 3GT | UDP-rhamnose | peonidin-3-O-rhamnoside |
| Peonidin | 3GT | UDP-xylose | peonidin-3-O-xyloside |
| Peonidin | 5GT | UDP-arabinose | peonidin-5-O-arabinoside |
| Peonidin | 5GT | UDP-galactose | peonidin-5-O-galactoside |
| Peonidin | 5GT | UDP-glucose | peonidin-5-O-glucoside |
| Peonidin | 5GT | UDP-rhamnose | peonidin-5-O-rhamnoside |
| Peonidin | 5GT | UDP-xylose | peonidin-5-O-xyloside |
| Petunidin | 3GT | UDP-arabinose | petunidin-3-O-arabinoside |
| Petunidin | 3GT | UDP-galactose | petunidin-3-O-galactoside |
| Petunidin | 3GT | UDP-glucose | petunidin-3-O-glucoside |
| Petunidin | 3GT | UDP-rhamnose | petunidin-3-O-rhamnoside |
| Petunidin | 3GT | UDP-xylose | petunidin-3-O-xyloside |
| Petunidin | 5GT | UDP-arabinose | petunidin-5-O-arabinoside |
| Petunidin | 5GT | UDP-galactose | petunidin-5-O-galactoside |
| Petunidin | 5GT | UDP-glucose | petunidin-5-O-glucoside |
| Petunidin | 5GT | UDP-rhamnose | petunidin-5-O-rhamnoside |
| Petunidin | 5GT | UDP-xylose | petunidin-5-O-xyloside |

The starting materials and reactants for preparation of anthocyanins from anthocyanidins may be obtained from commercial sources or by readily available synthetic processes from available starting materials. For example, cyanidin, delphinidin, malvidin, pelargonidin, peonidin, and petunidin are commercially available, e.g., from ChromaDex, Inc.

The methods disclosed herein may be used to prepare mono-, di-, tri-, tetra-, and penta-, hexa- and poly-substituted anthocyanins. In order to make di-, tri-, tetra-, penta-, hexa- and higher substituted anthocyanins, multiple synthetic steps may be carried in the provided cell free system. A starting material for an n-glycosylated anthocyanin may be an n-1 glycosylated anthocyanin. For example, to make cyanidin-3,5-O-diglucoside, the anthocyanidin is first converted to cyanidin-3-O-glucoside (C3G) by a 3-glycotransferase (3GT) enzyme, and this C3G molecule is subsequently modified by a 5-glycotransferase (5GT) enzyme to add a second glucosyl moiety at position R⁵ to make the final cyanidin-3,5-O-glucoside. Addition of glycosyl groups by 3GT enzymes is generally the initial step toward n-glycosylated anthocyanins, as C3G anthocyanins are the most basic anthocyanin building blocks. Successive modification of C3G molecules by non-glucosyl donors can include but is not limited to acylation, malonylation, coumaroylation, caffeoylation, feruloylation.

Table 3 sets forth the anthocyanin products and their likely corresponding anthocyanidin and glycosyl donor starting materials, along with the predicted enzymes necessary to carry out the corresponding reactions.

TABLE 3
Anthocyanidin and glycosyl donor molecules and the corresponding anthocyanin
products using multiple glycotransferase enzymes and glycosylation steps

| | | | Glycosyl Donor |
|---|---|---|---|
| Anthocyanidin | Enzyme | Anthocyanin Product | Molecule(s) |
| Cyanidin | 3-O- | cyanidin-3-O-(6″-acetyl)- | uridine-5′- |
| | glycosyltransferase | galactoside | diphosphate- |
| | | | galactose |
| Cyanidin | 3-O- | cyanidin-3-O- | uridine-5′- |
| | glycosyltransferase | sambubioside | diphosphate-glucose |
| | | | & uridine-5′- |
| | | | diphosphate-xylose |
| Cyanidin | 3-O- | cyanidin-3-O-(3″- | uridine-5′- |
| | glycosyltransferase | malonyl)-glucoside | diphosphate-glucose |
| Cyanidin | 3-O- | cyanidin-3-O-(6″- | uridine-5′- |
| | glycosyltransferase | malonyl)-glucoside | diphosphate-glucose |
| Cyanidin | 3-O- | cyanidin-3-O-(6″- | uridine-5′- |
| | glycosyltransferase | dioxaloyl)-glucoside | diphosphate-glucose |
| Cyanidin | 3-O- | cyanidin-3-O-rutinoside | uridine-5′- |
| | glycosyltransferase; 3- | | diphosphate-glucose |
| | O-glucoside-6″-O- | | & uridine-5′- |
| | rhamnosyltransferase | | diphosphate- |
| | (3RT) | | rhamnose |
| Cyanidin | 3-O- | cyanidin-3-O- | uridine-5′- |
| | glycosyltransferase; 3- | xylosylrutinoside | diphosphate-glucose, |
| | O-glucoside-6″-O- | | uridine-5′- |
| | rhamnosyltransferase | | diphosphate- |
| | (3RT) | | rhamnose, uridine- |
| | | | 5′-diphosphate |
| | | | xylose |
| Cyanidin | 3-O- | cyanidin-3- | uridine-5′- |
| | glycosyltransferase; 3- | glucosylrutinoside | diphosphate-glucose |
| | O-glucoside-6″-O- | | & uridine-5′- |
| | rhamnosyltransferase | | diphosphate- |
| | (3RT) | | rhamnose |
| Cyanidin | 3-O- | cyanidin-3,5-O- | uridine-5′- |
| | glycosyltransferase; 5- | diglucoside | diphosphate-glucose |
| | O-glycosyltransferase | | |
| Cyanidin | 3-O- | cyanidin-3-O- | uridine-5′- |
| | glycosyltransferase; 3- | sophoroside | diphosphate-glucose |
| | O-glucoside-2″-O- | | |
| | glucotransferase | | |
| | (3GGT) | | |
| Cyanidin | 3-O- | cyanidin-3-O-(6-O-p- | uridine-5′- |
| | glycosyltransferase | coumaroyl)-glucoside | diphosphate-glucose |
| Cyanidin | 3-O- | cyanidin-3-O-(6-O- | uridine-5′- |
| | glycosyltransferase | malonylglucoside) | diphosphate-glucose |
| Cyanidin | 3-O- | cyanidin-3-O-3″,6″-O- | uridine-5′- |
| | glycosyltransferase | dimalonylglucoside | diphosphate-glucose |
| Cyanidin | 3-O- | cyanidin-3-(6-p- | uridine-5′- |
| | glycosyltransferase | caffeoyl)-glucoside | diphosphate-glucose |
| Cyanidin | 3-O- | cyanidin-3,5-O- | uridine-5′- |
| | glycosyltransferase; 5- | dihexoside | diphosphate-glucose |
| | O-glycosyltransferase | | |
| Cyanidin | 3-O- | cyanidin-3-O-lathyroside | uridine-5′- |
| | glycosyltransferase | | diphosphate- |
| | | | galactose & uridine- |
| | | | 5′-diphosphate- |
| | | | xylose |
| Cyanidin | 3-O- | cyanidin-3,3′-diglucoside | uridine-5′- |
| | glycosyltransferase; 3′- | | diphosphate-glucose |
| | O-glycosyltransferase | | |
| Cyanidin | 3-O- | cyanidin-3-O-xylosyl- | uridine-5′- |
| | glycosyltransferase | galactoside | diphosphate- |
| | | | galactose & uridine- |
| | | | 5′-diphosphate- |
| | | | xylose |
| Cyanidin | 3-O- | cyanidin-3-O- | uridine-5′- |
| | glycosyltransferase | (6″malonylglucoside) | diphosphate-glucose |
| Cyanidin | 3-O- | cyanidin-3-O- | uridine-5′- |
| | glycosyltransferase | dimalonylglucoside | diphosphate-glucose |
| Cyanidin | 3-O- | cyanidin-3-O- | uridine-5′- |
| | glycosyltransferase | malonylglucoside | diphosphate-glucose |
| Cyanidin | 3-O- | cyanidin-3-O- | uridine-5′- |
| | glycosyltransferase | succinylglucoside | diphosphate-glucose |
| Delphinidin | 3-O- | delphinidin-3-O- | uridine-5′- |
| | glycosyltransferase | sambubioside | diphosphate-glucose |
| | | | & uridine-5′- |
| | | | diphosphate-xylose |
| Delphinidin | 3-O- | delphinidin-3,5-O- | uridine-5′- |
| | glycosyltransferase; 5- | diglucoside | diphosphate-glucose |
| | O-glycosyltransferase | | |
| Delphinidin | 3-O- | delphinidin-3-O- | uridine-5′- |
| | glycosyltransferase; 3- | rutinoside | diphosphate-glucose |
| | O-glucoside-6″-O- | | & uridine-5′- |
| | rhamnosyltransferase | | diphosphate- |
| | (3RT) | | rhamnose |
| Delphinidin | 3-O- | delphinidin-3,5-O- | uridine-5′- |
| | glycosyltransferase; 5- | diglucoside | diphosphate-glucose |
| | O-glycosyltransferase | | |
| Delphinidin | 3-O- | delphinidin-3-O- | uridine-5′- |
| | glycosyltransferase; 3- | sophoroside | diphosphate-glucose |
| | O-glucoside-2″-O- | | |
| | glucotransferase | | |
| | (3GGT) | | |
| Delphinidin | 3-O- | delphinidin-3-O- | uridine-5′- |
| | glycosyltransferase; 5- | sambubioside-5-O- | diphosphate-glucose |
| | O-glycosyltransferase | glucoside | & uridine-5′- |
| | | | diphosphate-xylose |
| Delphinidin | 3-O- | delphinidin-3-O-(6″-O- | uridine-5′- |
| | glycosyltransferase | malonyl-glucoside) | diphosphate-glucose |
| Delphinidin | 3-O- | delphinidin-3-O-(6- | uridine-5′- |
| | glycosyltransferase | caffeoylglucoside) | diphosphate-glucose |
| Delphinidin | 3-O- | delphinidin-3-O-(6-p- | uridine-5′- |
| | glycosyltransferase | coumaroyl)-glucoside | diphosphate-glucose |
| Delphinidin | 3-O- | delphinidin-3,7-O- | uridine-5′- |
| | glycosyltransferase; 7- | diglucoside | diphosphate-glucose |
| | O-glycosyltransferase | | |
| Malvidin | 3-O- | malvidin-3,5-O- | uridine-5′- |
| | glycosyltransferase; 5- | diglucoside | diphosphate-glucose |
| | O-glycosyltransferase | | |
| Malvidin | 3-O- | malvidin-3-O- | uridine-5′- |
| | glycosyltransferase | acetylglucoside | diphosphate-glucose |
| Malvidin | 3-O- | malvidin-3-p- | uridine-5′- |
| | glycosyltransferase | coumaroylglucoside | diphosphate-glucose |
| Malvidin | 3-O- | malvin | uridine-5′- |
| | glycosyltransferase; 5- | | diphosphate-glucose |
| | O-glycosyltransferase | | |
| Pelargonidin | 3-O- | pelargonidin-3,5-O- | uridine-5′- |
| | glycosyltransferase; 5- | diglucoside | diphosphate-glucose |
| | O-glycosyltransferase | | |
| Pelargonidin | 3-O- | pelargonidin-3-O- | uridine-5′- |
| | glycosyltransferase; 3- | rutinoside | diphosphate-glucose |
| | O-glucoside-6″-O- | | & uridine-5′- |
| | rhamnosyltransferase | | diphosphate- |
| | (3RT) | | rhamnose |
| Pelargonidin | 3-O- | pelargonidin-3-O- | uridine-5′- |
| | glycosyltransferase | acetylglucoside | diphosphate-glucose |
| Pelargonidin | 3-O- | pelargonidin-3-O- | uridine-5′- |
| | glycosyltransferase | malylglucoside | diphosphate-glucose |
| Pelargonidin | 3-O- | pelargonidin-3-(6-p- | uridine-5′- |
| | glycosyltransferase | coumaroyl)-glucoside | diphosphate-glucose |
| Pelargonidin | 3-O- | pelargonidin-3-O-(6″-O- | uridine-5′- |
| | glycosyltransferase | malonylglucoside) | diphosphate-glucose |
| Pelargonidin | 3-O- | pelargonidin-3-O- | uridine-5′- |
| | glycosyltransferase | dimalonylglucoside | diphosphate-glucose |
| Pelargonidin | 3-O- | pelargonidin-3-O- | uridine-5′- |
| | glycosyltransferase; 3- | sophoroside | diphosphate-glucose |
| | O-glucoside-2″-O- | | |
| | glucotransferase | | |
| | (3GGT) | | |
| Pelargonidin | 3-O- | pelargonidin-3-O-(6- | uridine-5′- |
| | glycosyltransferase | caffeoylglucoside) | diphosphate-glucose |
| Peonidin | 3-O- | peonidin-3-O-rutinoside | uridine-5′- |
| | glycosyltransferase; 3- | | diphosphate-glucose |
| | O-glucoside-6″-O- | | & uridine-5′- |
| | rhamnosyltransferase | | diphosphate- |
| | (3RT) | | rhamnose |
| Peonidin | 3-O- | peonidin-3-O- | uridine-5′- |
| | glycosyltransferase | acetylglucoside | diphosphate-glucose |
| Peonidin | 3-O- | peonidin-3-p- | uridine-5′- |
| | glycosyltransferase | coumaroylglucoside | diphosphate-glucose |
| Peonidin | 3-O- | peonidin-3-O-(6″- | uridine-5′- |
| | glycosyltransferase | malonylglucoside) | diphosphate-glucose |
| Peonidin | 3-O- | peonidin-3-O- | uridine-5′- |
| | glycosyltransferase | dimalonylglucoside | diphosphate-glucose |
| Petunidin | 3-O- | petunidin-3-O-(6-O-p- | uridine-5′- |
| | glycosyltransferase | coumaroyl glucoside) | diphosphate-glucose |
| Cyanidin | 3-O- | cyanidin-3,5,3'-O- | uridine-5′- |
| | glycosyltransferase; 5- | triglucoside | diphosphate-glucose |
| | O-glycosyltransferase | | |
| Cyanidin | 3-O- | cyanidin-3-O-(6-O-p- | uridine-5′- |
| | glycosyltransferase; 5- | coumaroyl)-glucoside-5- | diphosphate-glucose |
| | O-glycosyltransferase | O-glucoside | |
| Cyanidin | 3-O- | cyanidin-3-O-rutinoside- | uridine-5′- |
| | glycosyltransferase; 5- | 5-O-glucoside | diphosphate-glucose |
| | O-glycosyltransferase; | | & uridine-5′- |
| | 3-O-glucoside-6″-O- | | diphosphate- |
| | rhamnosyltransferase | | rhamnose |
| | (3RT) | | |
| Cyanidin | 3-O- | cyanidin-3-O-(6″-O- | uridine-5′- |
| | glycosyltransferase | malonyl-2″-O- | diphosphate-glucose |
| | | glucuronyl)-glucoside | |
| Cyanidin | 3-O- | cyanidin-3-(p- | uridine-5′- |
| | glycosyltransferase; 5- | coumaroyl)-rutinoside-5- | diphosphate-glucose |
| | O-glycosyltransferase; | glucoside | & uridine-5′- |
| | 3-O-glucoside-6″-O- | | diphosphate- |
| | rhamnosyltransferase | | rhamnose |
| | (3RT) | | |
| Cyanidin | 3-O- | cyanidin-3-glucoside-5- | uridine-5′- |
| | glycosyltransferase; 5- | caffeoylglucoside | diphosphate-glucose |
| | O-glycosyltransferase | | |
| Cyanidin | 3-O- | malonylshisonin | uridine-5′- |
| | glycosyltransferase; 5- | | diphosphate-glucose |
| | O-glycosyltransferase | | |
| Cyanidin | 3-O- | cyanidin-3-O- | uridine-5′- |
| | glycosyltransferase; 5- | sambubioside-5-O- | diphosphate-glucose, |
| | O-glycosyltransferase | rhamnoside | uridine-5′- |
| | | | diphosphate-xylose, |
| | | | uridine-5′- |
| | | | diphosphate- |
| | | | rhamnose |
| Cyanidin | 3-O- | cyanidin-3-O- | uridine-5′- |
| | glycosyltransferase; 5- | sophoroside-5-O- | diphosphate-glucose |
| | O-glycosyltransferase; | rhamnoside | & uridine-5′- |
| | 3-O-glucoside-2″-O- | | diphosphate- |
| | glucotransferase | | rhamnose |
| | (3GGT) | | |
| Cyanidin | 3-O- | cyanidin-3-O- | uridine-5′- |
| | glycosyltransferase; 5- | sambubioside-5-O- | diphosphate-glucose |
| | O-glycosyltransferase | glucoside | & uridine-5′- |
| | | | diphosphate-xylose |
| Cyanidin | 3-O- | cyanidin-3-O-(caffeoyl)- | uridine-5′- |
| | glycosyltransferase | diglucose | diphosphate-glucose |
| Cyanidin | 3-O- | cyanidin-3-O-(cis-p- | uridine-5′- |
| | glycosyltransferase | coumaroyl)-diglucoside | diphosphate-glucose |
| Cyanidin | 3-O- | cyanidin-3-O-(trans-p- | uridine-5′- |
| | glycosyltransferase | coumaroyl)-diglucoside | diphosphate-glucose |
| Cyanidin | 3-O- | cyanidin-3-O- | uridine-5′- |
| | glycosyltransferase; 5- | diglucoside-5-O- | diphosphate-glucose |
| | O-glycosyltransferase | glucoside | |
| Cyanidin | 3-O- | cyanidin-3-O-xylosyl- | uridine-5′- |
| | glycosyltransferase | glucosyl-galactoside | diphosphate-glucose, |
| | | | uridine-5′- |
| | | | diphosphate-xylose, |
| | | | uridine-5′- |
| | | | diphosphate- |
| | | | galactose |
| Cyanidin | 3-O- | cyanidin-3-O- | uridine-5′- |
| | glycosyltransferase; 5- | malonylglucoside-5- | diphosphate-glucose |
| | O-glycosyltransferase | glucoside | |
| Cyanidin | 3-O- | cyanidin-3-(caffeoyl | uridine-5′- |
| | glycosyltransferase; 5- | sophoroside)-5-glucoside | diphosphate-glucose |
| | O-glycosyltransferase; | | |
| | 3-O-glucoside-2″-O- | | |
| | glucotransferase | | |
| | (3GGT) | | |
| Cyanidin | 3-O- | cyanidin-3-O- | uridine-5′- |
| | glycosyltransferase; 5- | sophoroside-5-O- | diphosphate-glucose |
| | O-glycosyltransferase; | glucoside | |
| | 3-O-glucoside-2″-O- | | |
| | glucotransferase | | |
| | (3GGT) | | |
| Delphinidin | 3-O- | delphinidin-3-O- | uridine-5′- |
| | glycosyltransferase; 5- | sambubioside-5-O- | diphosphate-glucose |
| | O-glycosyltransferase | glucoside | & uridine-5′- |
| | | | diphosphate-xylose |
| Delphinidin | 3-O- | delphinidin-3-O- | uridine-5′- |
| | glycosyltransferase | (caffeoyl)-diglucoside | diphosphate-glucose |
| Delphinidin | 3-O- | delphinidin-3-O- | uridine-5′- |
| | glycosyltransferase; 5- | (caffeoyl)-rutinoside-5- | diphosphate-glucose |
| | O-glycosyltransferase; | O-glucoside | & uridine-5′- |
| | 3-O-glucoside-6″-O- | | diphosphate- |
| | | | rhamnose |
| | rhamnosyltransferase | | |
| | (3RT) | | |
| Delphinidin | 3-O- | delphinidin-3-O- | uridine-5′- |
| | glycosyltransferase; 5- | (feruloyl)-rutinoside-5- | diphosphate-glucose |
| | O-glycosyltransferase; | O-glucoside | & uridine-5′- |
| | 3-O-glucoside-6″-O- | | diphosphate- |
| | rhamnosyltransferase | | rhamnose |
| | (3RT) | | |
| Delphinidin | 3-O- | delphinidin-3-O- | uridine-5′- |
| | glycosyltransferase; 5- | glucoside-5-caffoyl- | diphosphate-glucose |
| | O-glycosyltransferase | glucoside | |
| Delphinidin | 3-O- | delphinidin-3-O- | uridine-5′- |
| | glycosyltransferase; 5- | glucoside-5-O-(6- | diphosphate-glucose |
| | O-glycosyltransferase | coumaroylglucoside) | |
| Delphinidin | 3-O- | delphinidin-3-O-(6″-O- | uridine-5′- |
| | glycosyltransferase; 3′- | malonyl)-glucoside-3′-O- | diphosphate-glucose |
| | O-glycosyltransferase | glucoside | |
| Delphinidin | 3-O- | delphinidin-3-O-3″,6″-O- | uridine-5′- |
| | glycosyltransferase | dimalonylglucoside | diphosphate-glucose |
| Delphinidin | 3-O- | delphinidin-3,5,3′-O- | uridine-5′- |
| | glycosyltransferase; 3′- | triglucoside | diphosphate-glucose |
| | O-glycosyltransferase; | | |
| | 5-O- | | |
| | glycosyltransferase | | |
| Pelargonidin | 3-O- | pelargonidin-3-O- | uridine-5′- |
| | glycosyltransferase; 5- | rutinoside-5-O-glucoside | diphosphate-glucose |
| | O-glycosyltransferase; | | & uridine-5′- |
| | 3-O-glucoside-6″-O- | | diphosphate- |
| | rhamnosyltransferase | | rhamnose |
| | (3RT) | | |
| Pelargonidin | 3-O- | pelargonidin-3-O-(p- | uridine-5′- |
| | glycosyltransferase | coumaroyl)-diglucoside | diphosphate-glucose |
| Pelargonidin | 3-O- | pelargonidin-3-O-3″,6″- | uridine-5′- |
| | glycosyltransferase | O-dimalonylglucoside | diphosphate-glucose |
| Pelargonidin | 3-O- | pelargonidin-3-O- | uridine-5′- |
| | glycosyltransferase; 5- | sambubioside-5-O- | diphosphate-glucose |
| | O-glycosyltransferase | glucoside | & uridine-5′- |
| | | | diphosphate-xylose |
| Pelargonidin | 3-O- | pelargonidin-3-O- | uridine-5′- |
| | glycosyltransferase; 5- | glucoside-5-O-(6- | diphosphate-glucose |
| | O-glycosyltransferase | coumaroylglucoside) | |
| Pelargonidin | 3-O- | pelargonidin-3- | uridine-5′- |
| | glycosyltransferase; 5- | glucoside-5- | diphosphate-glucose |
| | O-glycosyltransferase | caffeoylglucoside | |
| Pelargonidin | 3-O- | pelargonidin-3-O-(6- | uridine-5′- |
| | glycosyltransferase; 5- | caffeoylglucoside)-5-O- | diphosphate-glucose |
| | O-glycosyltransferase | glucoside | |
| Peonidin | 3-O- | peonidin-3-(6″-p- | uridine-5′- |
| | glycosyltransferase; 5- | coumaroyl-glucoside)-5- | diphosphate-glucose |
| | O-glycosyltransferase | glucoside | |
| Peonidin | 3-O- | peonidin-3-O- | uridine-5′- |
| | glycosyltransferase; 5- | sophoroside-5-O- | diphosphate-glucose |
| | O-glycosyltransferase; | glucoside | |
| | 3-O-glucoside-2″-O- | | |
| | glucotransferase | | |
| | (3GGT) | | |
| Cyanidin | 3-O- | cyanidin-3-xylosyl- | uridine-5′- |
| | glycosyltransferase | glucosyl-galactoside- | diphosphate-glucose, |
| | | coumaric acid | uridine-5′- |
| | | | diphosphate-xylose, |
| | | | uridine-5′- |
| | | | diphosphate- |
| | | | galactose |
| Cyanidin | 3-O- | cyanidin-3-xylosyl- | uridine-5′- |
| | glycosyltransferase | glucosyl-galactoside- | diphosphate-glucose, |
| | | ferulic acid | uridine-5′- |
| | | | diphosphate-xylose, |
| | | | uridine-5′- |
| | | | diphosphate- |
| | | | galactose |
| Cyanidin | 3-O- | cyanidin-3-xylosyl- | uridine-5′- |
| | glycosyltransferase | glucosyl-galactoside- | diphosphate-glucose, |
| | | sinapic acid | uridine-5′- |
| | | | diphosphate-xylose, |
| | | | uridine-5′- |
| | | | diphosphate- |
| | | | galactose |
| Cyanidin | 3-O- | cyanidin-3-(caffeoyl- | uridine-5′- |
| | glycosyltransferase; 5- | feruloyl sophoroside)-5- | diphosphate-glucose |
| | O-glycosyltransferase; | glucoside | |
| | 3-O-glucoside-2″-O- | | |
| | glucotransferase | | |
| | (3GGT) | | |
| Cyanidin | 3-O- | cyanidin-3-(caffeoyl-p- | uridine-5′- |
| | glycosyltransferase; 5- | hydroxybenzoyl | diphosphate-glucose |
| | O-glycosyltransferase; | sophoroside)-5-glucoside | |
| | 3-O-glucoside-2″-O- | | |
| | glucotransferase | | |
| | (3GGT) | | |
| Cyanidin | 3-O- | cyanidin-3- | uridine-5′- |
| | glycosyltransferase; 5- | (dicaffeoylsophoroside)- | diphosphate-glucose |
| | O-glycosyltransferase; | 5-glucoside | |
| | 3-O-glucoside-2″-O- | | |
| | glucotransferase | | |
| | (3GGT) | | |
| Cyanidin | 3-O- | cyanidin-3-(feruloyl | uridine-5′- |
| | glycosyltransferase; 5- | sophoroside)-5-glucoside | diphosphate-glucose |
| | O-glycosyltransferase; | | |
| | 3-O-glucoside-2″-O- | | |
| | glucotransferase | | |
| | (3GGT) | | |
| Cyanidin | 3-O- | cyanidin-3-(p- | uridine-5′- |
| | glycosyltransferase; 5- | hydroxybenzoylsophoroside)- | diphosphate-glucose |
| | O-glycosyltransferase; | 5-glucoside | |
| | 3-O-glucoside-2″-O- | | |
| | glucotransferase | | |
| | (3GGT) | | |
| Cyanidin | 3-O- | cyanidin-3-(caffeoyl)(p- | uridine-5′- |
| | glycosyltransferase; 5- | coumaroyl)-diglucoside- | diphosphate-glucose |
| | O-glycosyltransferase | 5-glucoside | |
| Cyanidin | 3-O- | cyanidin-3-(sinapoyl)- | uridine-5′- |
| | glycosyltransferase; 5- | diglucoside-5-glucoside | diphosphate-glucose |
| | O-glycosyltransferase | | |
| Cyanidin | 3-O- | cyanidin-3-(p- | uridine-5′- |
| | glycosyltransferase; 5- | coumaroyl)-diglucoside- | diphosphate-glucose |
| | O-glycosyltransferase | 5-glucoside | |
| Delphinidin | 3-O- | delphinidin-3-O-(trans- | uridine-5′- |
| | glycosyltransferase; 5- | coumaroyl)-rutinoside-5- | diphosphate-glucose |
| | O-glycosyltransferase; | glucoside | & uridine-5′- |
| | 3-O-glucoside-6″-O- | | diphosphate- |
| | rhamnosyltransferase | | rhamnose |
| | (3RT) | | |
| Delphinidin | 3-O- | delphinidin-3,3′- | uridine-5′- |
| | glycosyltransferase; 3′- | diglucoside-5-(6- | diphosphate-glucose |
| | O-glycosyltransferase; | caffeoyl)-glucoside | |
| | 5-O- | | |
| | glycosyltransferase | | |
| Delphinidin | 3-O- | delphinidin-3-O-(p- | uridine-5′- |
| | glycosyltransferase; 5- | coumaroyl)-rutinoside-5- | diphosphate-glucose |
| | O-glycosyltransferase; | O-glucoside | & uridine-5′- |
| | 3-O-glucoside-6″-O- | | diphosphate- |
| | rhamnosyltransferase | | rhamnose |
| | (3RT) | | |
| Malvidin | 3-O- | malvidin-3-(feruloyl)- | uridine-5′- |
| | glycosyltransferase; 5- | rutinoside-5-glucoside | diphosphate-glucose |
| | O-glycosyltransferase; | | & uridine-5′- |
| | 3-O-glucoside-6″-O- | | diphosphate- |
| | rhamnosyltransferase | | rhamnose |
| | (3RT) | | |
| Malvidin | 3-O- | malvidin-3-(p- | uridine-5′- |
| | glycosyltransferase; 5- | coumaroyl)-rutinoside-5- | diphosphate-glucose |
| | O-glycosyltransferase; | glucoside | & uridine-5′- |
| | 3-O-glucoside-6″-O- | | diphosphate- |
| | rhamnosyltransferase | | rhamnose |
| | (3RT) | | |
| Pelargonidin | 3-O- | pelargonidin-3-O-(6-O- | uridine-5′- |
| | glycosyltransferase; 5- | caffeoylglucoside)-5-O- | diphosphate-glucose |
| | O-glycosyltransferase | (6-O-malonylglucoside) | |
| | | (monodemalonylsalvianin) | |
| Peonidin | 3-O- | peonidin-3-(caffeoyl | uridine-5′- |
| | glycosyltransferase; 5- | sophoroside)-5-glucoside | diphosphate-glucose |
| | O-glycosyltransferase; | | |
| | 3-O-glucoside-2″-O- | | |
| | glucotransferase | | |
| | (3GGT) | | |
| Peonidin | 3-O- | peonidin-3-(feruloyl | uridine-5′- |
| | glycosyltransferase; 5- | sophoroside)-5-glucoside | diphosphate-glucose |
| | O-glycosyltransferase; | | |
| | 3-O-glucoside-2″-O- | | |
| | glucotransferase | | |
| | (3GGT) | | |
| Peonidin | 3-O- | peonidin-3-(p- | uridine-5′- |
| | glycosyltransferase; 5- | hydroxybenzoylsophoroside)- | diphosphate-glucose |
| | O-glycosyltransferase; | 5-glucoside | |
| | 3-O-glucoside-2″-O- | | |
| | glucotransferase | | |
| | (3GGT) | | |
| Peonidin | 3-O- | peonidin-3-(p- | uridine-5′- |
| | glycosyltransferase; 5- | coumaroyl)-rutinoside-5- | diphosphate-glucose |
| | O-glycosyltransferase; | glucoside | & uridine-5′- |
| | 3-O-glucoside-6″-O- | | diphosphate- |
| | rhamnosyltransferase | | rhamnose |
| | (3RT) | | |
| Petunidin | 3-O- | petunidin-3-(feruloyl)- | uridine-5′- |
| | glycosyltransferase; 5- | rutinoside-5-glucoside | diphosphate-glucose |
| | O-glycosyltransferase; | | & uridine-5′- |
| | 3-O-glucoside-6″-O- | | diphosphate- |
| | rhamnosyltransferase | | rhamnose |
| | (3RT) | | |
| Petunidin | 3-O- | petunidin-3-(p- | uridine-5′- |
| | glycosyltransferase; 5- | coumaroyl)-rutinoside-5- | diphosphate-glucose |
| | O-glycosyltransferase; | glucoside | & uridine-5′- |
| | 3-O-glucoside-6″-O- | | diphosphate- |
| | rhamnosyltransferase | | rhamnose |
| | (3RT) | | |
| Cyanidin | 3-O- | cyanidin-3-(caffeoyl- | uridine-5′- |
| | glycosyltransferase; 5- | sinapoyl)-diglucoside-5- | diphosphate-glucose |
| | O-glycosyltransferase | glucoside | |
| Cyanidin | 3-O- | cyanidin-3-(p- | uridine-5′- |
| | glycosyltransferase; 5- | coumaroyl)(sinapoyl)- | diphosphate-glucose |
| | O-glycosyltransferase | triglucoside-5-glucoside | |
| Cyanidin | 3-O- | cyanidin-3- | uridine-5′- |
| | glycosyltransferase; 5- | (feruloyl)(sinapoyl)- | diphosphate-glucose |
| | O-glycosyltransferase | triglucoside-5-glucoside | |
| Cyanidin | 3-O- | cyanidin-3-(p- | uridine-5′- |
| | glycosyltransferase; 5- | coumaroyl)(sinapoyl)- | diphosphate-glucose |
| | O-glycosyltransferase | diglucoside-5-glucoside | |
| Cyanidin | 3-O- | cyanidin-3- | uridine-5′- |
| | glycosyltransferase; 5- | (feruloyl)(sinapoyl)- | diphosphate-glucose |
| | O-glycosyltransferase | diglucoside-5-glucoside | |
| Cyanidin | 3-O- | cyanidin-3- | uridine-5′- |
| | glycosyltransferase; 5- | (sinapoyl)(sinapoyl)- | diphosphate-glucose |
| | O-glycosyltransferase | diglucoside-5-glucoside | |
| Cyanidin | 3-O- | cyanidin-3-(sinapoyl)- | uridine-5′- |
| | glycosyltransferase; 5- | diglucoside-5-(sinapoyl)- | diphosphate-glucose |
| | O-glycosyltransferase | glucoside | |
| Delphinidin | 3-O- | delphinidin-3-O- | uridine-5′- |
| | glycosyltransferase; 3′- | glucosyl-5-O- | diphosphate-glucose |
| | O-glycosyltransferase; | caffeoylglucoside-3′-O- | |
| | 5-O- | caffeoylglucoside | |
| | glycosyltransferase | (gentiodelphin) | |
| Delphinidin | 3-O- | delphinidin-3-O-(6-O- | uridine-5′- |
| | glycosyltransferase; 5- | (trans-p-coumaroyl)- | diphosphate-glucose |
| | O-glycosyltransferase | glucosyl)-5-O-(6-O- | |
| | | malonylglucoside) | |
| | | (malonylawobanin) | |
| Pelargonidin | 3-O- | pelargonidin-3-(caffeoyl- | uridine-5′- |
| | glycosyltransferase; 5- | feruloyl sophoroside)-5- | diphosphate-glucose |
| | O-glycosyltransferase | O-glucoside | |
| Pelargonidin | 3-O- | monardaein | uridine-5′- |
| | glycosyltransferase; 5- | | diphosphate-glucose |
| | O-glycosyltransferase | | |
| Pelargonidin | 3-O- | pelargonidin-3-O-(6-O- | uridine-5′- |
| | glycosyltransferase; 5- | caffeoylglucoside)-5-O- | diphosphate-glucose |
| | O-glycosyltransferase | (4-O,6-O- | |
| | | dimalonylglucoside) | |
| | | (salvianin) | |
| Peonidin | 3-O- | peonidin-3-(caffeoyl- | uridine-5′- |
| | glycosyltransferase; 5- | feruloyl sophoroside)-5- | diphosphate-glucose |
| | O-glycosyltransferase; | glucoside | |
| | 3-O-glucoside-2″-O- | | |
| | glucotransferase | | |
| | (3GGT) | | |
| Peonidin | 3-O- | peonidin-3-(caffeoyl-p- | uridine-5′- |
| | glycosyltransferase; 5- | coumaroyl sophoroside)- | diphosphate-glucose |
| | O-glycosyltransferase; | 5-glucoside | |
| | 3-O-glucoside-2″-O- | | |
| | glucotransferase | | |
| | (3GGT) | | |
| Peonidin | 3-O- | peonidin-3-(caffeoyl-p- | uridine-5′- |
| | glycosyltransferase; 5- | hydroxybenzoylsophoroside)- | diphosphate-glucose |
| | O-glycosyltransferase; | 5-glucoside | |
| | 3-O-glucoside-2″-O- | | |
| | glucotransferase | | |
| | (3GGT) | | |
| Peonidin | 3-O- | peonidin-3- | uridine-5′- |
| | glycosyltransferase; 5- | (dicaffeoylsophoroside)- | diphosphate-glucose |
| | O-glycosyltransferase; | 5-glucoside | |
| | 3-O-glucoside-2″-O- | | |
| | glucotransferase | | |
| | (3GGT) | | |
| Peonidin | 3-O- | peonidin-3-(feruloyl-p- | uridine-5′- |
| | glycosyltransferase; 5- | coumaroyl sophoroside)- | diphosphate-glucose |
| | O-glycosyltransferase; | 5-glucoside | |
| | 3-O-glucoside-2″-O- | | |
| | glucotransferase | | |
| | (3GGT) | | |
| Peonidin | 3-O- | peonidin-3-(feruloyl-p- | uridine-5′- |
| | glycosyltransferase; 5- | hydroxybenzoylsophoroside)- | diphosphate-glucose |
| | O-glycosyltransferase; | 5-glucoside | |
| | 3-O-glucoside-2″-O- | | |
| | glucotransferase | | |
| | (3GGT) | | |
| Peonidin | 3-O- | peonidin-3-(2-(6-(3- | uridine-5′- |
| | glycosyltransferase; 5- | (glucosyl)caffeoyl)glucosyl)- | diphosphate-glucose |
| | O-glycosyltransferase | 6(4-(6-(3- | |
| | | (glucosyl)caffeoyl)glucosyl)caffeoyl)glucoside)- | |
| | | 5-glucoside (heavenly blue | |
| | | anthocyanin) | |

### Co-solvents

The reaction mixtures and reaction solutions may comprise co-solvents, i.e., solvents along with water. Various co-solvents may be used in the reaction solutions and reaction mixtures to improve solubility. In some embodiments, the co-solvent may comprise ab out 1 to ab out 75% (v/v) of the reaction solution or reaction mixture, e.g., about 5 to about 50% (v/v) or 10 to about 40% (v/v). The co-solvent may be, e.g., dimethylformamide (DMF), ethanol (EtOH), methanol (MeOH), dimethylsulfoxide (DMSO).

### Anthocyanin purity

The anthocyanin products may be produced in exceptional purity. For example, the anthocyanin products may be produced in greater than 70% purity, greater than 80% purity, greater than 90% purity, greater than 95% purity, greater than 97% purity, greater than 98% purity, greater than 99% purity, greater than 99.5% purity, or greater than 99.9% purity. Thus, purities in a range of 70 to 99.9% purity, 80 to 99.9% purity, 90 to 99.9% purity, 95 to 99.9% purity can be achieved.

### EXAMPLE

The following is a non-limiting example of the present invention. It is to be understood that said example is not intended to limit the present invention in any way. Equivalents or substitutes are within the scope of the present invention.

### Enzyme Expression and Purification

All genes were synthesized and cloned into expression plasmids and then transformed into E. coli cells for expression. Cells were grown in TB media supplemented with 50 μg/mL kanamycin sulfate at 37° C. and 200 rpm until A₆₀₀=0.6. Cells were cooled to 18° C., expression was induced and grown for an additional 18 h. Cell pellets were collected by centrifugation, frozen, and then resuspended in a 5 mL lysis buffer (50 mM sodium phosphate pH 7.5, 300 mM NaCl, 5 mM imidazole) per gram of cell paste. Cell lysates were prepared by sonication and cellular debris was removed by centrifugation. Clarified lysate was loaded onto GE XK series columns containing IMAC-Nickel resin. Proteins were eluted using a 15CV gradient from buffer A (50 mM sodium phosphate pH 7.5, 300 mM NaCl, 10% glycerol (w/v)) into 25% buffer B (1 M imidazole, 50 mM sodium phosphate pH 7.5, 300 mM NaCl, 10% glycerol (w/v)). Fractions containing proteins of interest were pooled and exchanged into 50 mM sodium phosphate pH 7.5, 10% glycerol (w/v), and 0.1 mM EDTA with a GE HiPrep 26/10 desalting column. Enzymes were then concentrated with Amicon spin filtration units to a value of 5 mg/mL, mixed with 15% glycerol (w/v) and flash frozen.

A skilled artisan would understand that E. coli cells are exemplary to demonstrate the concept of the present invention. A skilled artisan would understand that other suitable cells, including bacteria, for example, bacillus subtillis, or fungi such as trichoderma or aspergillus terrus, or any other host cell suitable for expressing these genes are within the scope of the present invention.

### Analytical Methods

For sampling, the reaction fluid was acidified with 2M HCl (1:10 v/v), followed by high-speed centrifugation and filtration through 0.45 μm filters. Samples were run on an HPLC system to examine the amount of cyanidin and glycosylated cyanidin present in the reaction mixture. The HPLC method was as follows: An Agilent 1200 HPLC was fitted with a Ascentis C18 HPLC column 150 mm×4.6, 3 um. The column was heated to 25C with the sample block being maintained at 15C. For each sample, 10 uL was injected and the product was eluted at a flow rate of 1.0 ml/min using 0.1% phosphoric acid in water (solvent A) and acetonitrile (solvent B) with the following gradient: 90% A to 50% A for 6 min, 90% A for 0.1 min, and 90% A for 2.9 min for column equilibration. The run time was a total of 9 minutes with cyanidin-3-glucoside eluting at 3.8 min and cyanidin eluting at 4.6 min. A diode array detector (DAD) was used for the detection of the molecule of interest at 530 nm.

### C3G Production with 3GT Enzymes and Reaction Optimization

As described herein, cyanidin is glycosylated by the 3-O-glycotransferase enzyme (3GT, 2.4.2.51) to form cyanidin-3-glucoside (C3G). Although this 3GT enzyme family is found in plants and shown to be active in microbial hosts, a significant advancement is needed to produce industrially relevant amounts of C3G. Enzymes from Table 4 were expressed, purified and screened for activity to generate C3G from cyanidin. Enzymes were initially screened for optimal values for substrate concentration, pH, temperature, buffering agent, and time. The stability and solubility of the substrate cyanidin was found to be limiting and therefore additional optimization of co-solvent type and amount in these reactions was also required. The reaction solution (100 mM sodium acetate, pH 5.0, 20 mM UDP-glucose, 5 mM cyanidin, 5 mM MgCl₂, 20% DMF (v/v)) was mixed with 20 μM enzyme at 30° C. for 30 minutes. Yield has been up to 1.6 g/L.

### Immobilized 3-O-glycotransferase to Create Cyanidin-3-glucoside

After demonstrating and optimizing generation of C3G from cyanidin with free enzyme, the next step was to immobilize 3GT enzymes onto solid supports in an effort to increase stability, longevity, and catalysis. Different commercial support materials were screened for product and substrate retention, enzyme retention, and activity of the immobilized enzyme. The support collection comprised of various surface chemistries for the following types of linkage: covalent, adsorption, ionic, affinity, encapsulation, and entrapment. Typically, 50 mg of resin was mixed with 4.0 mg of enzyme in buffer for 16-24 h at room temperature. The amount of immobilized enzyme was quantified by measuring protein concentration in solution before and after immobilization by either BCA or Bradford assay. The amount of starting material and product retained on the resin was quantified by HPLC.

Following initial resin screening the best enzyme-support combination was selected for further optimization. Immobilized enzymes were again subject to various reaction conditions (changes in substrate concentration, pH, temperature, buffering agent, solvent, time) to ensure optimal activity.

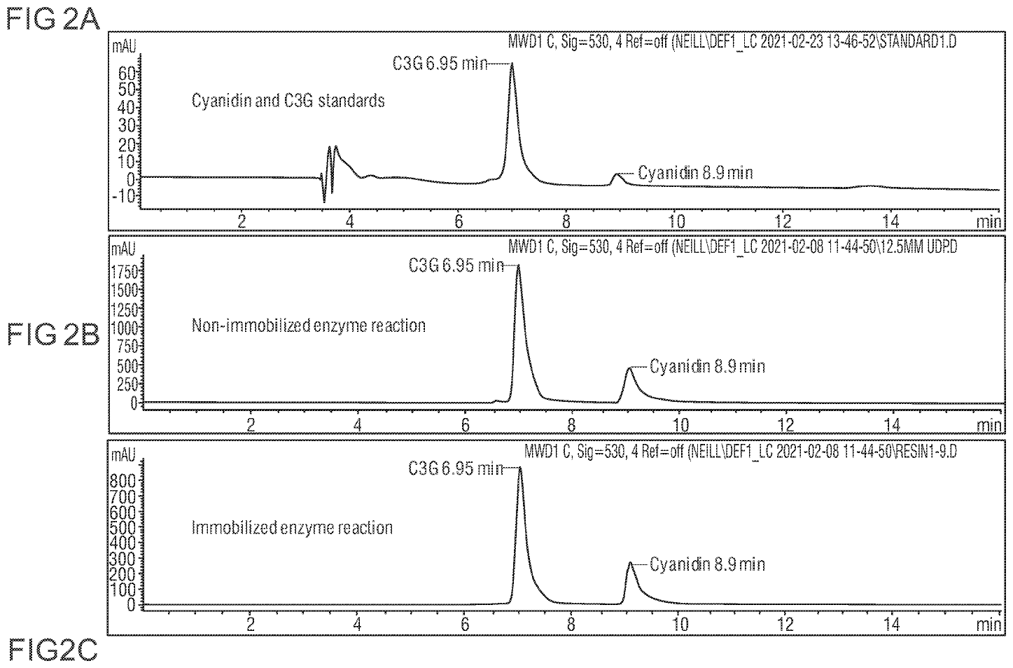

3GT was mixed with epoxy methacrylate resin. Enzyme immobilized resin was then incubated with 10 mg/ml polyethyleneimine (PEI) for at least 1 h. Immobilized enzyme was used to convert cyanidin into C3G. Reaction solution (100 mM sodium acetate, pH 5.0, 15 mM UDP-glucose, 4 mM cyanidin, 40% DMF (v/v)) was mixed with 2.5 mg immobilized enzyme at 30° C. for 30 min. Immobilized 3GT from Vitis labrusca was able to convert 2.9 mM cyanidin for a yield of 2.1 mM C3G (51.4%, 0.924 g/L) (FIG. 2C).

### Use of Immobilized 3GT Create C3G in a Continuous Reactor

A reactor of 2.75″ length with a 0.125″ outside diameter and a 0.055″ internal diameter containing immobilized Vl3GT enzyme (2.7 mg enzyme on 50 mg of resin) was heated to 30° C. and equilibrated for 30 minutes with equilibration buffer (50 mM sodium acetate, pH 5.0) by pumping it through the reactor. After this time, the substrate solution was flowed through the reactor at a flow rate of 2.3 μL/min. Prior to entering the reactor, the substrate solution was generated by mixing 400 μL of 4.0 mM cyanidin at a flow rate of 0.93 μL/min with 600 μL of 15 mM UDP-glucose, 100 mM sodium acetate, pH 5.0 at a flow rate of 1.38 μL/min to yield a total flow rate through the reactor of 2.3 μL/min (30-minute residence time). After the solution has passed through the reactor, the fluid was collected and sampled by HPLC for the presence of cyanidin and C3G formation. The immobilized Vl3GT enzyme produced C3G at 0.795 g/L in the continuous reactor.

As used herein, the term “about” refers to plus or minus 10% of the referenced number.

TABLE 4
Glycosyltransferase Enzyme Sequences:

| | | SEQ |
|---|---|---|
| | | ID |
| Enzyme: | Sequence: | NO: |
| flavonoid | MSQTTTNPHVAVLAFPFSTHAAPLLAVVRRLAV | 1 |
| 3-O- | AAPHAVFSFFSTSESNASIFHDSMHTMQCNIKS | |
| glycosyl- | YDVSDGVPEGYVFTGRPQEGIDLFMRAAPESFR | |
| transferase | QGMVMAVAETGRPVSCLVADAFIWFAADMAAEM | |
| Vitis | GVAWLPFWTAGPNSLSTHVYIDEIREKIGVSGI | |
| labrusca | QGREDELLNFIPGMSKVRFRDLQEGIVFGNLNS | |
| Accession: | LFSRLLHRMGQVLPKATAVFINSFEELDDSLTN | |
| ABR24135 | DLKSKLKTYLNIGPFNLITPPPVVPNTTGCLQW | |
| | LKERKPTSVVYISFGTVTTPPPAELVALAEALE | |
| | ASRVPFIWSLRDKARMHLPEGFLEKTRGHGMVV | |
| | PWAPQAEVLAHEAVGAFVTHCGWNSLWESVAGG | |
| | VPLICRPFFGDQRLNGRMVEDVLEIGVRIEGGV | |
| | FTKSGLMSCFDQILSQEKGKKLRENLRALRETA | |
| | DRAVGPKGSSTENFKTLVDLVSKPKDV | |
| flavonoid | MSQTTTNPHVAVLAFPFSTHAAPLLAVVRRLAA | 2 |
| 3-O- | AAPHAVFSFFSTSQSNASIFHDSMHTMQCNIKS | |
| glycosyl- | YDISDGVPEGYVFAGRPQEDIELFTRAAPESFR | |
| transferase | QGMVMAVAETGRPVSCLVADAFIWFAADMAAEM | |
| Vitis | GLAWLPFWTAGPNSLSTHVYIDEIREKIGVSGI | |
| vinifera | QGREDELLNFIPGMSKVRFRDLQEGIVFGNLNS | |
| Accession: | LFSRMLHRMGQVLPKATAVFINSFEELDDSLTN | |
| P51094 | DLKSKLKTYLNIGPFNLITPPPVVPNTTGCLQW | |
| | LKERKPTSVVYISFGTVTTPPPAEVVALSEALE | |
| | ASRVPFIWSLRDKARVHLPEGFLEKTRGYGMVV | |
| | PWAPQAEVLAHEAVGAFVTHCGWNSLWESVAGG | |
| | VPLICRPFFGDQRLNGRMVEDVLEIGVRIEGGV | |
| | FTKSGLMSCFDQILSQEKGKKLRENLRALRETA | |
| | DRAVGPKGSSTENFITLVDLVSKPKDV | |
| flavonoid | MVFQSHIGVLAFPFGTHAAPLLTVVQRLATSSP | 3 |
| 3-O- | HTLFSFFNSAVSNSTLFNNGVLDSYDNIRVYHV | |
| glycosyl- | WDGTPQGQAFTGSHFEAVGLFLKASPGNFDKVI | |
| transferase | DEAEVETGLKISCLITDAFLWFGYDLAEKRGVP | |
| Scutellaria | WLAFWTSAQCALSAHMYTHEILKAVGSNGVGET | |
| baicalensis | AEEELIQSLIPGLEMAHLSDLPPEIFFDKNPNP | |
| Accession: | LAITINKMVLKLPKSTAVILNSFEEIDPIITTD | |
| MK577650 | LKSKFHHFLNIGPSILSSPTPPPPDDKTGCLAW | |
| | LDSQTRPKSVVYISFGTVITPPENELAALSEAL | |
| | ETCNYPFLWSLNDRAKKSLPTGFLDRTKELGMI | |
| | VPWAPQPRVLAHRSVGVFVTHCGWNSILESICS | |
| | GVPLICRPFFGDQKLNSRMVEDSWKIGVRLEGG | |
| | VLSKTATVEALGRVMMSEEGEIIRENVNEMNEK | |
| | AKIAVEPKGSSFKNFNKLLEIINAPQSS | |
| flavonoid | MGVFGSNESSSMSIVMYPWLAFGHMTPFLHLSN | 4 |
| 3-O- | KLAEKGHKIVFLLPKKALNQLEPLNLYPNLITF | |
| glycosyl- | HTISIPQVKGLPPGAETNSDVPFFLTHLLAVAM | |
| transferase | DQTRPEVETIFRTIKPDLVFYDSAHWIPEIAKP | |
| Arabidopsis | IGAKTVCFNIVSAASIALSLVPSAEREVIDGKE | |
| thaliana | MSGEELAKTPLGYPSSKVVLRPHEAKSLSFVWR | |
| Accession: | KHEAIGSFFDGKVTAMRNCDAIAIRTCRETEGK | |
| Q9LVW3 | FCDYISRQYSKPVYLTGPVLPGSQPNQPSLDPQ | |
| | WAEWLAKFNHGSVVFCAFGSQPVVNKIDQFQEL | |
| | CLGLESTGFPFLVAIKPPSGVSTVEEALPEGFK | |
| | ERVQGRGVVFGGWIQQPLVLNHPSVGCFVSHCG | |
| | FGSMWESLMSDCQIVLVPQHGEQILNARLMTEE | |
| | MEVAVEVEREKKGWFSRQSLENAVKSVMEEGSE | |
| | IGEKVRKNHDKWRCVLTDSGFSDGYIDKFEQNL | |
| | IELVKS | |
| UDP- | MGSTNLEPHVAVLVFPFATHAGLLFGLVQRLAK | 5 |
| galactose: | AAPNVKFTFFNTAKSNHSSFSNSSSIASNVIPY | |
| cyanidin | DVYDGVEEGYVFSGKPQEDINLFLAVAADEFRR | |
| 3-O- | GLEKAVVDSGRQISCLVADAFLWFSCDLAQEIG | |
| galactosyl- | VPWVPLWTSGACSLSTHIYTDLIRQTVGFDGIE | |
| transferase | GRMDEKLNFIPGYSNLRLGDLPGGVVFGNLESP | |
| 1 | FSVMLHKMGQVLPRADVLVMNSFEELDPDLMKD | |
| Daucus | LSSKFKKILNVGPFNLTSPPASQYSDEYGCIPW | |
| carota | LDKRNPKSVAYIGFGTVAMLPPNEIVELAEALE | |
| Accession: | SSGTPFVWSLKDQSKKHLPEGFLERTRESGKIV | |
| KP319022 | AWAPQVQVLSHNAVGIVITHGGWNSVLESIAAG | |
| | VPLICRPFFGDHAINTWMVENVWKIGVRISGGV | |
| | FTKKGTADALEQVLLRQKGKELNQQITLLKDLA | |
| | FKAVGPNGSSSLNFTELVKVIAV | |

Although there has been shown and described some preferred embodiments of the present invention, it will be readily apparent to those skilled in the art that modifications may be made thereto which do not exceed the scope of the appended claims. Therefore, the scope of the invention is only to be limited by the following claims. The figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

### Bibliography

Feng et al., “Advances in engineering UDP-sugar supply for recombinant biosynthesis of glycosides in microbes,” Biotechnology Advances, https://doi.org/10.1016/j.biotechadv. 2020.107538, (2020).

Shrestha, et al., “Combinatorial approach for improved cyanidin 3-O-glucoside production in Escherichia coli,” Microb. Cell Fact. 18(7) (2019).

Yan, et al., “High-Yield Anthocyanin Biosynthesis in Engineered Escherichia coli, Biotech. and Bioeng. 100(1) (2008).

1. A method of producing an anthocyanin from an anthocyanidin, the method comprising:
a) adding to a mixture comprising a suitable solvent (i) an anthocyanidin of Formula I: 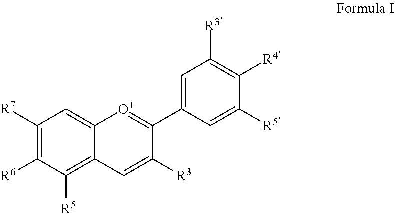
wherein each of R³, R⁵, R⁶, R⁷, R³′, R⁴′, and R⁵′ are independently selected from the group consisting of H, OH, and OCH₃, wherein at least one of R³, R⁵, R⁶, R⁷, R³′, R⁴′, and R⁵′ is OH or CH₃ and at least one of R³, R⁵, R⁶, R⁷, R³′, R⁴ ′, and R⁵′ is OH;
(ii) a glycosylated uridine-5′-diphosphate of Formula II: 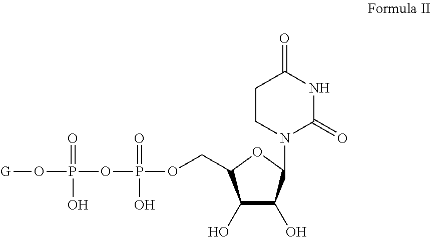
wherein G is a glycosyl residue selected from the group consisting of 5- and 6-membered monosaccharides; and
(iii) a glycosyltransferase enzyme to form a reaction mixture;
b) removing the supernatant from the reaction mixture from (a); and
c) isolating the anthocyanin.

2. The method of claim 1, wherein the anthocyanidin of Formula I comprises a member of the group consisting of cyanidin, delphinidin, malvidin, pelargonidin, peonidin and petunidin.

3. The method of claim 1, wherein G in Formula II comprises a member of the group consisting of glucose, galactose, xylose, arabinose, and rhamnose.

4. The method of claim 1 or claim 2, wherein the glycosylated uridine-5′-diphosphate of Formula II has a concentration in the reaction mixture of about 1 mM to about 30 mM.

5. The method of one of claims 1 to 4, wherein the glycosyltransferase enzyme comprises a 3-O-glycosyltransferase or a 5-O-glycosyltransferase.

6. The method of one of claims 1 to 4, wherein the glycosyltransferase enzyme comprises a member of the group consisting of 3-O-glucoside-6″-O-rhamnosyltransferase (3RT), 3-O-5 glucoside-2″-O-glucotransferase (3GGT), 3′-O-glycosyltransferase, or 7-O-glycosyltransferase.

7. The method of one of claims 1 to 6, wherein the sequence for the glycosyltransferase enzymes is one of SEQ ID NO 1-5, or an amino acid sequence with at least 90%, at least 92.5%, at least95%, at least 97.5%, at least99%, at least99.5%, or at least99.9% similarity to SEQ ID NO 1-5.

8. The method of one of claims 1 to 7, wherein the glycosyltransferase enzyme is non-immobilized.

9. The method of one of claims 1 to 7, wherein the glycosyltransferase enzyme is immobilized.

10. The method of claims 8 and 9, wherein the glycosyltransferase enzyme is housed in a continuous reactor system.

11. The method of one of claims 1 to 10, wherein R³ is OH, and the glycosyltransferase enzyme is 3-O-glycotrasferase (3GT).

12. The method of one of claims 1 to 10, wherein R⁵ is OH, and the glycosyltransferase enzyme is 5-O-glycotransferase (5GT).

13. The method of one of claims 1 to 12, wherein the glycosylated uridine-5′-diphosphate of Formula II is selected from the group consisting of UDP-arabinose, UDP-galactose, UDP-glucose, UDP-rhamnose, and UDP-xylose.

14. A method of one of claims 1 to 14 having a starting pH, wherein the starting pH is about 4 to about 6.

15. A method of producing an anthocyanin from an anthocyanidin, comprising:
a) in a cell-free vessel, providing a glycosyltransferase enzyme;
b) adding an anthocyanidin and a glycosylated uridine diphosphate to the cell-free vessel to form the anthocyanin; and
c) removing the anthocyanin from the cell-free vessel.

16. The method of claim 15, wherein the anthocyanidin is a member of the group consisting of cyanidin, delphinidin, malvidin, pelargonidin, peonidin and petunidin.

17. The method of claim 15 or claim 16, wherein G in Formula II is a member of the group consisting of glucose, galactose, xylose, arabinose, and rhamnose.

18. The method of one of claims 15 to 17, wherein the glycosylated uridine-5′-diphosphate of Formula II has a concentration in the reaction mixture of about 1 mM to about 30 mM.

19. The method of one of claims 15 to 18, wherein the glycosyltransferase enzyme is a 3-O-glycosyltransferase or 5-O-glycosyltransferase.

20. The method of one of claims 15 to 19, wherein the glycosyltransferase enzyme is a member of the group consisting of 3-O-glucoside-6″-O-rhamnosyltransferase (3RT), 3-O-glucoside-2″-O-glucotransferase (3GGT), 3′-O-glycosyltransferase, or 7-O-glycosyltransferase.

21. The method of one of claims 15 to 19, wherein the sequence for the glycosyltransferase enzymes is one of SEQ ID NO 1-5, or an amino acid sequence with at least 90%, at least

92. 5%, atleast95%, atleast 97.5%, atleast99%, atleast99.5%, or atleast99.9% similarity to SEQ ID NO 1-5.

22. The method of one of claims 15 to 20, wherein the glycosyltransferase enzyme is non-immobilized.

23. The method of one of claims 15 to 20, wherein the glycosyltransferase enzyme is immobilized.

24. The method of claims 22 and 23, wherein the glycosyltransferase enzyme is housed in a continuous reactor system.

25. The method of one of claims 15 to 24, wherein the glycosylated uridine-5′-diphosphate is selected from the group consisting of UDP-arabinose, UDP-galactose, UDP-glucose, UDP-rhamnose, and UDP-xylose.

26. The method of one of claims 15-25, wherein the cell-free vessel has a starting pH of about 4 to about 6.

27. A method of producing an anthocyanin from an anthocyanidin, the method comprising:
a) adding an anthocyanidin or anthocyanin of Formula Ia: 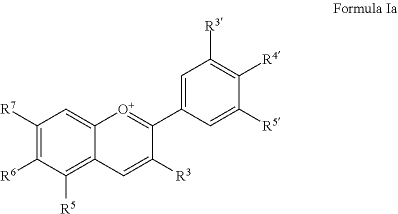
wherein each of R³, R⁵, R⁶, R⁷, R³′, R⁴′, and R⁵′ are independently selected from the group consisting of H, OH, OCH₃, or a sugar, wherein at least one of R³, R⁵, R⁶, R₇, R³′, R⁴′, and R₅′ is OH;
a glycosylated uridine-5′-diphosphate of Formula II: 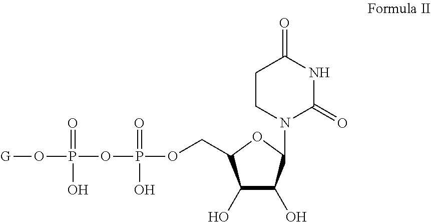
wherein G is a glycosyl residue selected from the group consisting of 5- and 6-membered monosaccharides; and
a glycosyltransferase enzyme to form a reaction mixture;
b) removing the supernatant from the reaction mixture from (a); and
c) isolating the anthocyanin.

28. The method of claim 27, wherein the anthocyanidin of Formula Ia is a member of the group consisting of cyanidin, delphinidin, malvidin, pelargonidin, peonidin, petunidin, a glycosylated cyanidin, a glycosylated delphinidin, a glycosylated malvidin, a glycosylated pelargonidin, and a glycosylated peonidin.

29. The method of claim 27, wherein the anthocyanidin of Formula Ia is selected from a glycosylated cyanidin, a glycosylated delphinidin, a glycosylated malvidin, a glycosylated pelargonidin, and a glycosylated peonidin.

30. The method of claim 27, wherein the anthocyanidin of Formula Ia is selected from a mono-, di-, tri-, tetra- or penta-glycosylated cyanidin, a mono-, di-, tri-, tetra- or penta-glycosylated delphinidin, a mono-, di-, tri-, tetra- or penta-glycosylated malvidin, a mono- , di-, tri-, tetra- or penta-glycosylated pelargonidin, and a mono-, di-, tri-, tetra- or penta-glycosylated peonidin.

31. The method of one of claims 27 to 30, wherein Gin Formula II is a member of the group consisting of glucose, galactose, xylose, arabinose, and rhamnose.

32. The method of one of claims 27 to 31, wherein the glycosylated uridine diphosphate has an initial concentration of about 1 mM to about 30 mM in the reaction mixture.

33. The method of one of claims 27 to 32, wherein the glycosyltransferase enzyme is a 3-O-glycosyltransferase or 5-O-glycosyltransferase.

34. The method of one of claims 27 to 33, wherein the glycosyltransferase enzyme is a member of the group consisting of 3-O-glucoside-6″-O-rhamnosyltransferase (3RT), 3-O-glucoside-2″-O-glucotransferase (3GGT), 3′-O-glycosyltransferase, or 7-O-glycosyltransferase.

35. The method of one of claims 27 to 34, wherein the sequence for the glycosyltransferase enzymes is one of SEQ ID NO 1-5, or an amino acid sequence with at least 90%, at least 92.5%, at least 95%, at least 97.5%, at least 99%, at least 99.5%, or at least 99.9% similarity to SEQ ID NO 1-5.

36. The method of one of claims 27 to 35, wherein the reaction mixture has a starting pH, wherein the starting pH is about 4 to about 6.

37. The method of one of claims 27 to 36, wherein the glycosyltransferase enzyme is non-immobilized.

38. The method of one of claims 27 to 36, wherein the glycosyltransferase enzyme is immobilized.

39. The method of claims 37 and 38, wherein the glycosyltransferase enzyme is housed in a continuous reactor system.

40. The method of one of claims 27 to 39, wherein R³ is OH, and the glycosyltransferase enzyme is 3-O-glycotransferase (3GT).

41. The method one of claims 27 to 40, wherein R⁵ is OH, and the glycosyltransferase enzyme is 5-O-glycotransferase (5GT).

42. The method of one of claims 27 to 41, wherein the glycosylated uridine-5′-diphosphate is selected from the group consisting of UDP-arabinose, UDP-galactose, UDP-glucose, UDP-rhamnose, and UDP-xylose.

43. The method of one of claims 27-42, wherein the reaction mixture has a starting pH of about 4 to about 6.


SEQUENCE LISTING (5 sequences)

SEQ ID NO: 1 - protein, 456 aa, Vitis labrusca
MSQTTTNPHVAVLAFPFSTHAAPLLAVVRRLAVAAPHAVFSFFSTSESNASIFHDSMHTMQCNIKSYDVSDGVPEGYVFTGRPQEGIDLFMRAAPESFRQGMVMAVAETGRPVSCLVADAFIWFAADMAAEMGVAWLPFWTAGPNSLSTHVYIDEIREKIGVSGIQGREDELLNFIPGMSKVRFRDLQEGIVFGNLNSLFSRLLHRMGQVLPKATAVFINSFEELDDSLTNDLKSKLKTYLNIGPFNLITPPPVVPNTTGCLQWLKERKPTSVVYISFGTVTTPPPAELVALAEALEASRVPFIWSLRDKARMHLPEGFLEKTRGHGMVVPWAPQAEVLAHEAVGAFVTHCGWNSLWESVAGGVPLICRPFFGDQRLNGRMVEDVLEIGVRIEGGVFTKSGLMSCFDQILSQEKGKKLRENLRALRETADRAVGPKGSSTENFKTLVDLVSKPKDV

SEQ ID NO: 2 - protein, 456 aa, Vitis vinifera
MSQTTTNPHVAVLAFPFSTHAAPLLAVVRRLAAAAPHAVFSFFSTSQSNASIFHDSMHTMQCNIKSYDISDGVPEGYVFAGRPQEDIELFTRAAPESFRQGMVMAVAETGRPVSCLVADAFIWFAADMAAEMGLAWLPFWTAGPNSLSTHVYIDEIREKIGVSGIQGREDELLNFIPGMSKVRFRDLQEGIVFGNLNSLFSRMLHRMGQVLPKATAVFINSFEELDDSLTNDLKSKLKTYLNIGPFNLITPPPVVPNTTGCLQWLKERKPTSVVYISFGTVTTPPPAEVVALSEALEASRVPFIWSLRDKARVHLPEGFLEKTRGYGMVVPWAPQAEVLAHEAVGAFVTHCGWNSLWESVAGGVPLICRPFFGDQRLNGRMVEDVLEIGVRIEGGVFTKSGLMSCFDQILSQEKGKKLRENLRALRETADRAVGPKGSSTENFITLVDLVSKPKDV

SEQ ID NO: 3 - protein, 457 aa, Scutellaria baicalensis
MVFQSHIGVLAFPFGTHAAPLLTVVQRLATSSPHTLFSFFNSAVSNSTLFNNGVLDSYDNIRVYHVWDGTPQGQAFTGSHFEAVGLFLKASPGNFDKVIDEAEVETGLKISCLITDAFLWFGYDLAEKRGVPWLAFWTSAQCALSAHMYTHEILKAVGSNGVGETAEEELIQSLIPGLEMAHLSDLPPEIFFDKNPNPLAITINKMVLKLPKSTAVILNSFEEIDPIITTDLKSKFHHFLNIGPSILSSPTPPPPDDKTGCLAWLDSQTRPKSVVYISFGTVITPPENELAALSEALETCNYPFLWSLNDRAKKSLPTGFLDRTKELGMIVPWAPQPRVLAHRSVGVFVTHCGWNSILESICSGVPLICRPFFGDQKLNSRMVEDSWKIGVRLEGGVLSKTATVEALGRVMMSEEGEIIRENVNEMNEKAKIAVEPKGSSFKNFNKLLEIINAPQSS

SEQ ID NO: 4 - protein, 468 aa, Arabidopsis thaliana
MGVFGSNESSSMSIVMYPWLAFGHMTPFLHLSNKLAEKGHKIVFLLPKKALNQLEPLNLYPNLITFHTISIPQVKGLPPGAETNSDVPFFLTHLLAVAMDQTRPEVETIFRTIKPDLVFYDSAHWIPEIAKPIGAKTVCFNIVSAASIALSLVPSAEREVIDGKEMSGEELAKTPLGYPSSKVVLRPHEAKSLSFVWRKHEAIGSFFDGKVTAMRNCDAIAIRTCRETEGKFCDYISRQYSKPVYLTGPVLPGSQPNQPSLDPQWAEWLAKFNHGSVVFCAFGSQPVVNKIDQFQELCLGLESTGFPFLVAIKPPSGVSTVEEALPEGFKERVQGRGVVFGGWIQQPLVLNHPSVGCFVSHCGFGSMWESLMSDCQIVLVPQHGEQILNARLMTEEMEVAVEVEREKKGWFSRQSLENAVKSVMEEGSEIGEKVRKNHDKWRCVLTDSGFSDGYIDKFEQNLIELVKS

SEQ ID NO: 5 - protein, 452 aa, Daucus carota
MGSTNLEPHVAVLVFPFATHAGLLFGLVQRLAKAAPNVKFTFFNTAKSNHSSFSNSSSIASNVIPYDVYDGVEEGYVFSGKPQEDINLFLAVAADEFRRGLEKAVVDSGRQISCLVADAFLWFSCDLAQEIGVPWVPLWTSGACSLSTHIYTDLIRQTVGFDGIEGRMDEKLNFIPGYSNLRLGDLPGGVVFGNLESPFSVMLHKMGQVLPRADVLVMNSFEELDPDLMKDLSSKFKKILNVGPFNLTSPPASQYSDEYGCIPWLDKRNPKSVAYIGFGTVAMLPPNEIVELAEALESSGTPFVWSLKDQSKKHLPEGFLERTRESGKIVAWAPQVQVLSHNAVGIVITHGGWNSVLESIAAGVPLICRPFFGDHAINTWMVENVWKIGVRISGGVFTKKGTADALEQVLLRQKGKELNQQITLLKDLAFKAVGPNGSSSLNFTELVKVIAV